(19) United States
(12) Patent Application Publication
(10) Pub. No.: US 2011/0209708 A1
(43) Pub. Date: Sep. 1, 2011

(54) Ventilation Interface Device

(75) Inventor: David M. Rapoport, New York, NY (US)

(73) Assignee: New York University, New York, NY (US)

(21) Appl. No.: 13/031,849

(22) Filed: Feb. 22, 2011

Related U.S. Application Data
(60) Provisional application No. 61/308,357, filed on Feb. 26, 2010.

(51) Int. Cl. A61M 16/06

(52) U.S. Cl. 128/205.25

(57) ABSTRACT

A device comprises a harness configured to secure a mask over a respiratory orifice of a head of a patient; an inflatable cushion, at least a portion of the first inflatable cushion being adjoined to portion of the harness, the first inflatable cushion being positioned over a predetermined area including a portion of a buccinator muscle of a cheek of a patient; and an inflation lumen having a first end and a second end, the first end receiving pressurized air, the second end connected to the first inflatable cushion to provide pressurized air. When pressurized air is provided to the respiratory orifice via the mask, (a) the inflatable cushion is inflated with the pressurized air, (b) a tension between the mask and the head is increased and (c) the pressurized air in the inflatable cushion counters a bowing of the cheeks during inspiration.

### PRIORITY CLAIM

This application claims priority to U.S. Provisional Application Ser. No. 61/308,357 entitled “Ventilation Interface Device” filed Feb. 26, 2010, the entire disclosure of which is incorporated herein by reference.

### FIELD OF THE INVENTION

The present invention relates to interface technology and, more particularly, to an interface device for a mask and strap arrangement for use in non-invasive ventilation or Continuous Positive Airway Pressure (“CPAP”) treatment that adjusts the tension of the strap to prevent the bowing of cheeks when administering a positive airway pressure to a patient.

### BACKGROUND INFORMATION

Noninvasive CPAP and Noninvasive Positive Pressure Ventilation (“NPPV”) treatments have come into widespread use for the treatment of sleep apnea and during episodes of acute and chronic respiratory failure without using endotracheal intubation. All forms of such non-invasive positive pressure ventilation (“PPV”) procedures require that a mask be worn over a respiratory passage of a patient to provide an interface with a source of positive air pressure. The technology for attaching such a mask includes elastic straps which are configured to provide a constant tension to the mask throughout the period of patient treatment. The tension value is selected to avoid overtightening, which can cause pain, skin irritation (e.g., facial ulcers, pressure sores, etc.) and/or skin breakdown while also avoiding overloosening, which can cause gas pressure leakage, thus impairing an efficacy of the treatment.

### SUMMARY OF THE INVENTION

The present invention is directed to a device comprising a harness configured to secure a mask over a respiratory orifice of a head of a patient and a first inflatable cushion, at least a portion of the first inflatable cushion being adjoined to a portion of the harness, the first inflatable cushion being positioned over a buccinator muscle of a first cheek. The device also comprises an inflation lumen having a first end and a second end, the first end receiving pressurized air, the second end connected to the first inflatable cushion to provide pressurized air, wherein when pressurized air is provided to the respiratory orifice via the mask, the first inflatable cushion is inflated with the pressurized air and a tension between the mask and the head is increased, inflation of the first inflatable cushion countering a bowing of the cheeks during inspiration.

### BRIEF DESCRIPTION OF THE DRAWINGS

FIG. 1 shows an exemplary embodiment of a system according to the present invention.

### DETAILED DESCRIPTION

The present invention may be further understood with reference to the following description and the appended drawings, wherein like elements are referred to with the same reference numerals. The present invention is directed to a pair of inflatable cheek splinting cushions configured for placement over a region of the cheeks of a face of a patient located over the buccinator muscle. The exemplary cheek splinting cushions of the present invention may be employed with a ventilation interface device (“VID”) which may be used in conjunction with various systems (e.g., PAP, NPPV, etc.) designed to administer pressurized air and/or gas to a patient. Although embodiments of the present invention are described with respect to CPAP procedures, the present invention may also be employed for the treatment of any respiratory condition where a mask is used to administer an airway pressure including treatments for sleep apnea, hypopnea, snoring, somnolence, etc.

An exemplary system and method according to the present invention is directed to a cheek splinting cushion situated on a strap for an airway pressure-administering mask. The system of the present invention dynamically adjusts a tension of the mask strap against a region of the cheeks located over the buccinator muscle so that a pressure between the mask strap and a skin surface is optimized in a manner that counters a bowing of the cheeks during inspiration, as those skilled in the art will understand. Specifically, the buccinator is a thin quadrilateral muscle occupying the interval between the maxilla and the mandible at the side of the face. The skin overlying the buccinator often bows during inspiration due to a lack of bony support. Inflatable cheek splinting cushions according to the present invention are positioned to align with the buccinator muscle and are selectively inflated or deflated to affect a tension of the mask strap against the cheeks. In one embodiment, when a pressure being supplied to the respiratory orifice is minimal or absent, the cheek splinting cushions are deflated and, when a pressure being supplied to the respiratory orifice is increased, the cheek splinting cushions are inflated to increase the tension of the mask strap to counter a bowing of the cheeks and to prevent an air pressure leak. One particular benefit of the present invention is to allow a pressure in the mask strap to be released when a patient is undergoing a combination unattended study, which, as those skilled in the art will understand, requires a period of diagnostic monitoring when there is no pressure in the system followed by a period of therapy when the system is pressurized. That is, the present invention allows maximal comfort and optimal tension during both pressurized and non-pressurized conditions. Since the mask strap fits tightly against the patient during rest and expiration phases as well as during inspiration or ventilation phases, the mask can be worn for long periods of time while minimizing discomfort and harmful complications to the patient.

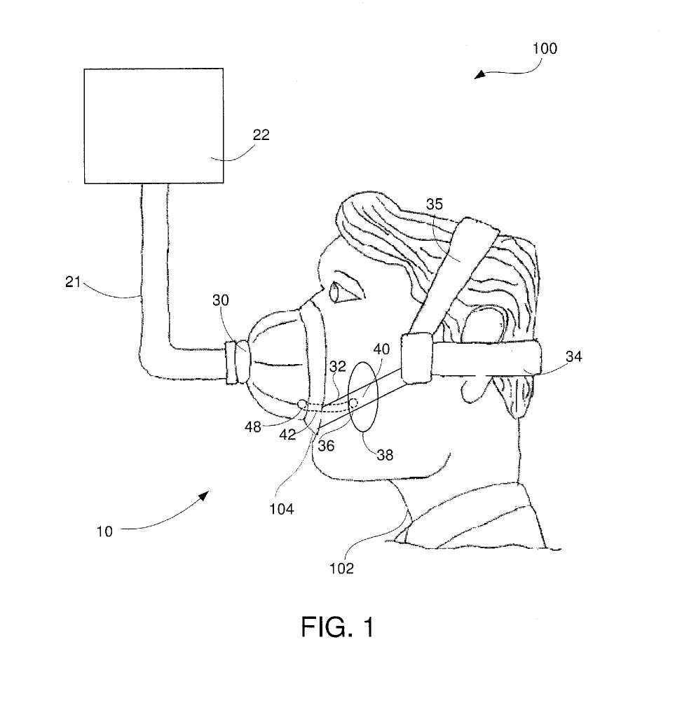

FIG. 1 shows an exemplary embodiment of a system 100 according to the present invention. The system 100 comprises a VID 10 which is connected via an air supply tube 21 to a flow generator 22 to receive air pressure therefrom. The flow generator 22 may deliver any of various flow patterns to the tubing 21 including, but not limited to, continuous pressure and varying high, low or zero pressures in synchrony with breathing, etc., as required by an exemplary treatment procedure for a patient. The VID 10 may include a mask 30 which, in an operative position, transfers the air pressure from the air supply tube 21 to a respiratory orifice 104 (i.e., one or both of the oral and nasal cavities) of a patient 102.

The mask 30 may be positioned over the respiratory orifice 104 of the patient 102 by a harness 32. The harness 32 may optionally comprise first and second straps 34, 35 which fit over and/or around the head of the patient 102, thereby securing the mask 30 thereto. The first and second straps 34, 35 may include a tightening mechanism (not shown) (e.g., a buckle, Velcro®, hooks, etc.) for sizing and/or tightening the harness 32 onto the head of the patient 102. It is further noted that any securing and tightening means may be employed to tighten the mask 30 onto the head of the patient without deviating from the scope of the present invention. In an exemplary embodiment, the tightening mechanism (not shown) is configured to maintain the integrity of an airtight seal between the face of the patient 102 and the mask 30, thereby preventing loss or leak of air pressure and movement of the mask 30. In one exemplary embodiment, the harness 32 must be configured to withstand a pressure exceeding 35 cm H₂O in accordance with an exemplary NPPV procedure without dislocating from a position over the respiratory orifice 104. In another exemplary embodiment, a gel may be used in conjunction with the mask 30 to enhance the seal and alleviate pressure on the face of the patient, as those skilled in the art will understand.

The VID 10 further comprises a set of cheek cushions 38 positioned to overlie the buccinator muscle of the patient. The cheek cushions 38 are attached at an attachment point 40 to the first strap 34 and extend away from the attachment point 40 by a predetermined distance selected to ensure a proper positioning over the buccinator muscle of the patient 102. In one exemplary embodiment, the cheek cushions 38 may be selectively positionable by a physician or other user of the system 100 after the VID has been attached to the patient 102. In the embodiment shown, the cheek cushions 38 are formed of a substantially circular or oval shape. It is noted however, that any shape (e.g., rectangular, etc.) and size of the cheek cushions 38 may be employed without deviating from the spirit and scope of the present invention.

Each of the cheek cushions 38 comprise openings 36 that open to an inflation lumen 42 extending a predetermined distance along the length of the first strap 34. In a first exemplary embodiment, the inflation lumen 42 connects the cheek cushions 38 to the air supply tube 21 via a connection 48. Specifically, the connection 48 opens into the mask 30 so that air supplied to the mask 30 via the air supply tube 21 also flows into the connection 48 to inflate the cheek cushions 38 fluidly connected thereto. Those of skill in the art would understand that the air pressure in the cheek cushions 38 will be substantially similar to that in the mask 30 and will cause the cheek cushions 38 to increase the tension on the mask 30 in proportion to the pressure in the mask 30 that is generated to ventilate the patient, but which also predisposes the mask 30 to leakage due to the bowing of the cheeks. Inflation of the cheek cushions 38 may further prevent a dissipation of the volume delivered to the respiratory orifice 104 of the patient 102. Each of the cheek cushions 38 are formed of a substantially flexible material with properties selected to affect an expansion thereof during inflation, as those skilled in the art will understand. In one exemplary embodiment, each of the cheek cushions 38 may be constructed of a thin rubber embedded within a firm cloth sheath that serves as the harness 32, or may be made of a non-distensible material whose shape determines the direction in which the cheek cushions 38 expand. It is further noted that although the inflation lumen 42 is depicted as extending out of the mask 30 and into the cheek cushion 38, the inflation lumen 42 may alternatively be formed integrally with the harness 32.

In accordance with an exemplary method of the present invention, the flow generator 22 supplies air pressure to the VID 10 via the air supply tube 21. As understood by those skilled in the art, the air pressure may have been determined when the inflation lumen 42 was not connected to the air supply tube 21. For example, a titration procedure performed on the patient may have indicated a maximum value of air pressure to be supplied to the patient 102. However, upon attachment of the inflation lumen 42 to the air supply tube 21, the air pressure that reaches the mask 30 may be intermittently or continuously reduced. Thus, the titration procedure is preferably performed after the inflation lumen 42 and cheek cushions 38 have been attached to the air supply tube 21.

During a period when no pressure is delivered to the patient airway (as during a period of wakefulness, diagnostic testing, and/or during brief transients as during the expiration phase of PPV), the air pressure supplied to the VID 10 by the flow generator 22 may have a lower value than the air pressure supplied during an inspiration phase of PPV (e.g., bilevel ventilation) or during periods of elevated CPAP, as those skilled in the art will understand. As such, during this period, the lower value of the air pressure may result in partial inflation or no inflation of the cheek cushions 38 relative to an initial state thereof. Furthermore, during this period of low pressure, the cheek cushions 38 may deflate relative to the initial state. Those skilled in the art would understand that the initial state of the cheek cushions 38 may be completely deflated or partially inflated. When the cheek cushions 38 are partially inflated or not inflated relative to the initial state (e.g., during any period of low pressure in the system), the patient 102 may not feel a pressure of the mask 30 on the face and, more specifically, over the buccinator muscle of the cheeks.

During the inspiration phase of a ventilation application, or during the sustained period of application of CPAP, an increased value of air pressure is supplied to the patient 102. When the air pressure reaches the mask 30, a first portion of the air pressure causes airflow to be transferred to the patient 102 via the respiratory orifice 104 and a second portion of the air pressure causes airflow into the inflation lumen 42 and subsequently into the cheek cushions 38. Thus, the patient 102 may receive air pressure and the cheek cushions 38 may inflate/deflate in a synchronized manner (e.g., inflation will occur during inspiration or high CPAP and deflation will occur during exhalation or low CPAP). As the cheek cushions 38 inflate, the mask 30 is pulled toward the face of the patient 102, thereby tightening the fit of the mask 30 on the face (i.e., increasing a tension between the mask 30 and the head of the patient 102). In this manner, the exemplary embodiment of the present invention counters the bowing of the cheeks during inspiration by increasing a volume of the cheek cushions 38.

In an alternate embodiment of the system 100, the cheek cushions 38 may be replaced with semi-rigid cheek cushions (not shown) that resemble a size and shape of the cheek cushions 38 in the inflated configuration. The cheek cushions may be secured to the first strap 34 and are configured to rest above the cheeks of the face to permit a splinting thereof. An attachment mechanism (e.g., a buckle, Velcro®, hooks, etc.) (not shown) may be provided on each of the cheek cushions to permit attachment to the harness 32. Alternatively, the cheek cushions may be permanently attached to or located within the first strap 34 of the harness 32 in a preset configuration. As those skilled in the art will understand, the cheek cushions (not shown) effectively counter a bowing of the cheeks during inspiration to prevent them from bowing out when pressure is applied to the respiratory airway of the patient.

While specific embodiments of the invention have been illustrated and described herein, it is realized that numerous modifications and changes will occur to those skilled in the art. It is therefore to be understood that the appended claims are intended to cover all such modifications and changes as fall within the true spirit and scope of the invention.

What is claimed is:

1. A device, comprising:
a harness configured to secure a mask over a respiratory orifice of a head of a patient;
an inflatable cushion, at least a portion of the first inflatable cushion being adjoined to a portion of the harness, the first inflatable cushion being positioned over a predetermined area including a portion of a buccinator muscle of a cheek of a patient; and
an inflation lumen having a first end and a second end, the first end receiving pressurized air, the second end connected to the first inflatable cushion to provide pressurized air,
wherein when pressurized air is provided to the respiratory orifice via the mask, (a) the inflatable cushion is inflated with the pressurized air, (b) a tension between the mask and the head is increased and (c) the pressurized air in the inflatable cushion counters a bowing of the cheeks during inspiration.

2. The device of claim 1, wherein, when no pressurized air is provided to the inflatable cushion, the inflatable cushion assumes a deflated shape and tension between the inflatable cushion and the buccinator muscle of the cheek is reduced.

3. The device of claim 1, further comprising an additional inflatable cushion positioned over a portion of a buccinator muscle of an additional cheek, inflation of the additional inflatable cushion countering bowing of the additional cheek during inspiration.

4. The device of claim 1, wherein the inflatable cushion is situated within a portion of the harness.

5. The device of claim 1, wherein the inflatable cushion is formed of a thin rubber material embedded within a firm cloth sheath.

6. The device according to claim 1, wherein the inflatable cushion is substantially rigid.

7. The device according to claim 1, wherein the inflatable cushion receives pressurized air from an airflow generator via an inflation lumen.

8. The device according to claim 7, wherein the inflation lumen is integral with the harness.

9. The device according to claim 7, wherein the inflation lumen extends out of the first bladder and connects directly to the airflow generator.

10. A system, comprising:
a mask which, when in an operative position, covers a respiratory opening of a patient;
a harness securing the mask to a head of the patient to maintain the mask in the operative position;
an inflatable bladder, at least a portion of the inflatable bladder being adjoined to a portion of the harness, the inflatable bladder being positioned over an buccinator muscle of a cheek of the patient; and
a pressure supply tubing coupled to the mask and providing pressurized air to the mask, wherein when pressurized air is received in the mask, the inflatable bladder is inflated and a tension between the inflatable bladder and the buccinator muscle of the patient is increased to counter a bowing of the cheeks during inspiration.

11. The system according to claim 10, further comprising an inflation lumen adjoined to the harness to fluidly connect the mask to the inflatable bladder.

12. The system according to claim 10, wherein the pressure supply tubing comprises a first tube for supplying pressurized air to the mask and a second tube for supplying pressurized air to the inflatable bladder.

13. The system according to claim 10, wherein the harness comprises at least one strap for securing the mask to the head of the patient.

14. The system of claim 10, further comprising an additional inflatable bladder positioned over a buccinator muscle of an additional cheek of the patient, wherein inflation of the additional inflatable bladder counters bowing of the additional cheek during inspiration.

15. A method for the treatment of a sleeping disorder in a patient, comprising:
providing a harness configured to secure a mask over a respiratory orifice of a head of the patient, a portion of the harness located over a buccinator muscle of a cheek of the patient comprising an inflatable cushion, the inflatable cushion being connected to an inflation lumen connected to a source of pressurized air;
supplying a positive airflow to a respiratory orifice of the patient during inspiration via a flow generator, the positive airflow flowing through the inflation lumen and into the inflatable cushion to cause an inflation thereof, inflation of the inflatable cushion countering a deflation of the cheek during inspiration to prevent a loosening of the harness from the head; and
termination the positive airflow during exhalation, the termination causing a deflation of the inflatable bladder.
